(19) United States
(12) Patent Application Publication
(10) Pub. No.: US 2005/0003525 A1
(43) Pub. Date: Jan. 6, 2005

(54) Stackable and pivotable petri dish system

(76) Inventor: Wei K. Hsu, Commerce, TX (US)

Correspondence Address: SHERMAN D PERNIA, ESQ., PC, 1110 NASA ROAD ONE, SUITE 450, HOUSTON, TX, 77058-3310, US

(21) Appl. No.: 10/835,235

(22) Filed: Apr. 29, 2004

Related U.S. Application Data
(60) Provisional application No. 60/466,252, filed on Apr. 29, 2003.
(60) Provisional application No. 60/473,107, filed on May 24, 2003.

(51) Int. Cl. C12M 1/22

(52) U.S. Cl. 435/305.1; 435/305.4

(57) ABSTRACT

In the present petri dish system the individual petri dishes are vertically stackable and pivotable relative to each other. An individual petri dish has a bottom or well member, a swivel support integral to and disposed adjacent the well member and a separate cover plate for covering the opening of the well on the bottom well member. The swivel support is adapted at its upper end to be received into the lower end of the swivel support of a second petri dish stacked on top of it. The individual petri dishes in a stack are pivotable relative to each other arround the axis of the stacked swivel supports. An optional interconnect rod may be used to secure the system in a stacked configuration.

### FIELD OF THE INVENTION

The present invention is in the field of molecular biology and microbiology. More specifically, the present invention relates to apparatuses useful in an in vitro process of measuring, testing and propagating microorganisms.

### BACKGROUND OF THE INVENTION

Petri dishes are known in the field, and generally have structures that are quite simple. On of the features desirable for petri dishes is that they be stackable for easy transport or storage. This stackability feature is usually accomplished by having top surface of the cover plate of the petri dish, the bottom surface of the dish itself, or both, be disposed to receive the the bottom or top respectively of an adjacent petri dish in the stack. Examples of such stacking strategies include U.S. Pat. Nos. 4,743,556 and 5,021,351, both to Ervin.

Although the petri dishes disclosed in these references may be useful for certain of their intended purpose, e.g., for stacking the dishes for storage, they may provide a less that satisfactory solution for transporting and handling such petri dishes. For example, the stacking means may not be positive or secure enough to handle and/or transport a sufficient plurality of individual at the same time. Also, if it is important to maintain the relative position of a specific petri dish in a stack during the handling of or working with the dishes, then additional care must be taken to make sure the individual dishes do not get mixed up.

In view of these considerations, it would be beneficial in the field to have an alternative petri dish system that provided a structurally more secure stacking means, and also provided structural means for maintaining the relative position of the petri dishes in a stack while they are being worked with.

### SUMMARY OF THE INVENTION

An object of the present invention is to provide an alternative petri dish and cover combination having the utility of certain typical prior petri dishes, but without certain of their disadvantages. More specifically, the present petri dish is intended to avoid the disadvantage of not being easily transportable. The transportability of the present petri dish and cover combination is accomplished at least in part by a stackability feature integral with the bottom member or well of the dish component of the combination.

The present petri dish system comprise individual petri dishes which are vertically stackable and pivotable relative to each other. An individual petri dish has a bottom or well member, a swivel support integral to and disposed adjacent the well member and a separate cover plate for covering the opening of the well on the bottom well member. The swivel support is adapted at its upper end to be received into the lower end of the swivel support of a second petri dish stacked on top of it. The individual petri dishes in a stack are pivotable relative to each other around the axis of the stacked swivel supports. A plurality of individual petri dishes of the present invention may be combined to form a stack as desired by the user.

An optional stacking rod is provided which may be used to secure the system in a stacked configuration. The stacking rod passes serially through an axial bore in each of the stacked swivel supports from bottom to top. The stacking rod stops within the axial bore of the bottom most petri dish, and after passing from the axial bore in the top most petri dish, a securing means is used on the rod to prevent the individual swivel support from separating from one another. The securing means can be as simple as the stacking rod being appropriately malleable and being bent over at the top of the swivel support stack.

The present petri dish system provides a structurally secure stacking means, especially when used with stacking rod means. Additionally, the present invention provides structural means for maintaining the relative positions of the individual petri dishes in a stack while they are being worked with.

### BRIEF DESCRIPTION OF THE DRAWINGS

FIG. 1 illustrates a stack of four of the present stackable and pivotable petri dishes.

FIG. 2A is a cross-section side elevation of the present stackable and pivotable petri dish showing its two separable components: a bottom member and a cover plate.

FIG. 2B is a top plan view of the bottom member of the present stackable and pivotable petri dish.

FIG. 2C is a cross-section side elevation of the swivel support of the present petri dish showing a swivel rod inserted through the lumen of the swivel support.

FIGS. 3A and 3B are top plan views of alternative configurations for a bottom member practicable in the present invention.

### DETAILED DESCRIPTION OF THE INVENTION

Referring now to the drawings, the details of preferred embodiments of the present invention are graphically and schematically illustrated. Like elements in the drawings are represented by like numbers, and any similar elements are represented by like numbers with a different lower case letter suffix.

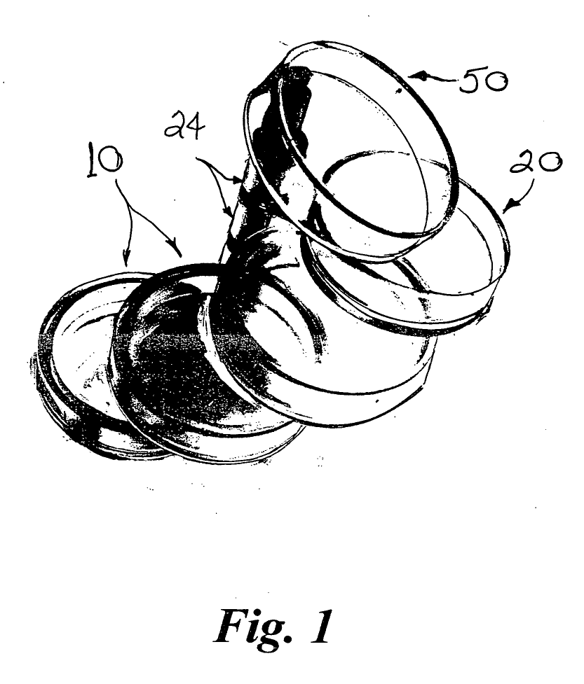

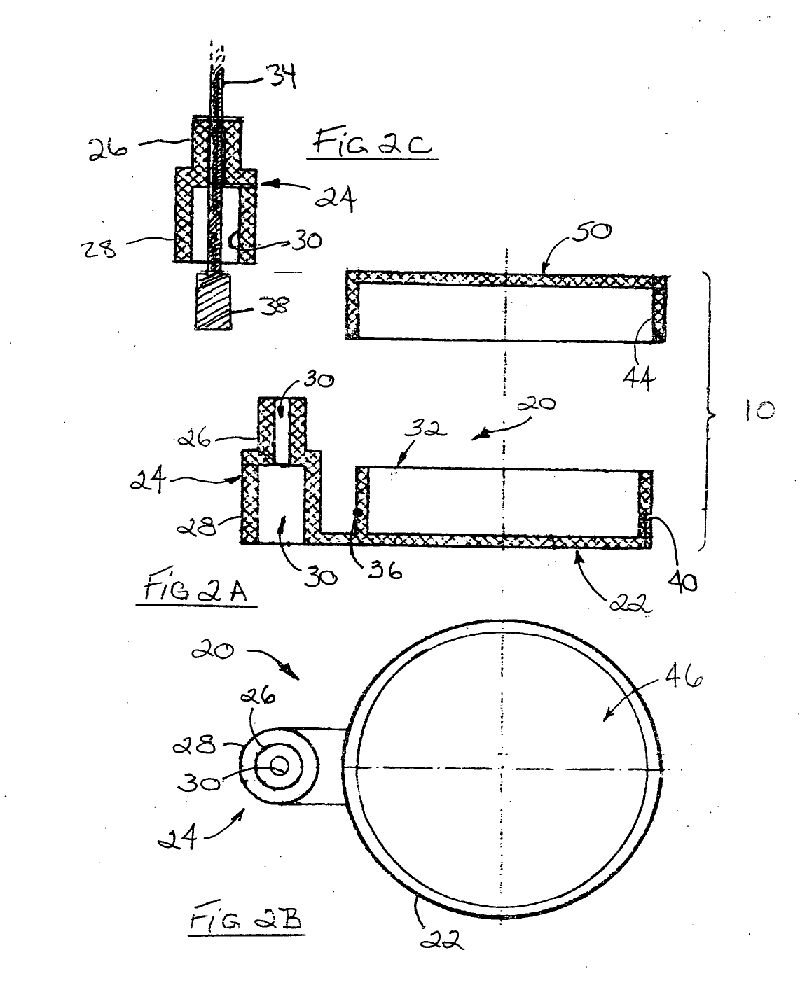

As shown in FIG. 1, the present stackable and pivotable petri dish 10 comprises two separable components: a bottom member 20 and a cover plate 50. As exemplified in FIGS. 2A and 2B, the bottom member 20 is further comprised of two inter-connected parts: a container or dish 22 and a swivel support 24. The dish 22 provides for containing a material or media such as a prior art petri dish might contain, such as a cell or tissue growth media. As illustrated in FIG. 2, the swivel support 24 of the bottom member 20 can be used to “nest” with the swivel support 24 of another bottom member 20. In this fashion, a plurality of bottom members 20 (with or without cover plates 50) can be stacked on top of each other in pivotable relationship.

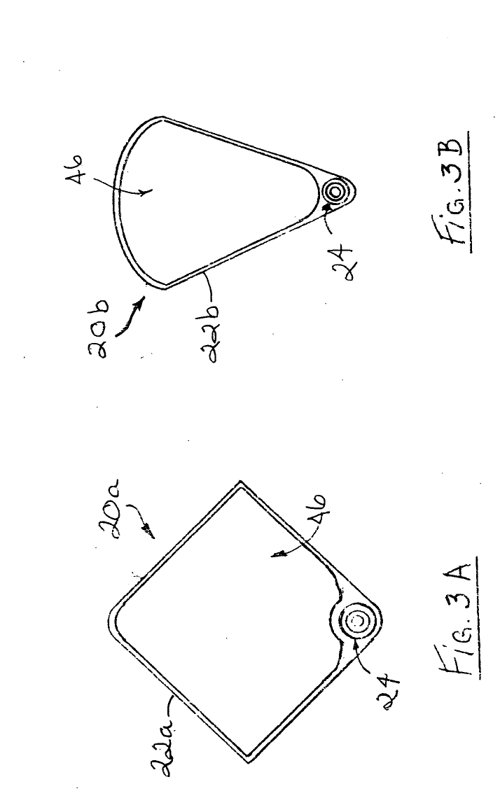

In a preferred embodiment as shown in FIG. 3, the upper neck 26 of the swivel support 24 on a first bottom member 20a was rotatably received into the axial bore or lumen 30 of the swivel support 24 at its lower barrel 28 on a second bottom member 20b. The axial bore or lumen 30 of the swivel support 24 passes concentrically through its lower barrel 28 and upper neck 26.

As exemplified in FIG. 2C, when a sufficient number of bottom members 20 are vertically stacked, an optional support or stacking rod 34 can be passed through the series of lumen 30 to provide added stability to the stack. Additionally, the support rod 34 can have a stop 38 at its lowed end, so that the upper end (not shown) of the support rod 34 extending beyond the series of lumen 30 can be used as a handle to securely transport a stack of the present petri dishes 10. When a petri dish unit 10 is ready, say for inoculation, the specific unit 10 is pivoted clear of the other units 10 in the stack, and the cover plate 50 is removed (under sterile conditions if necessary). Then the intended material is dispensed or inoculated into the dish 22 of the bottom member, and the dish 22 resealed with the cover plate 50.

In an alternative preferred embodiment, where it is desirable to have a particularly tight or close seal between the cover plate 50 and the dish 22 of the bottom member 20, an O-ring 36 is provided and seated in a notch 40 on the outer side 42 of the dish 22. The O-ring 36 engages the inner side 44 of the cover plate 50 when the cover plate 50 is installed over the dish opening 32. If an O-ring seal is used, the interior 46 of the dish 22 can be sealed from contaminating outside atmosphere.

In practicing the present stackable and pivotable petri dish in the embodiments of FIGS. 1 to 2C, the cover plate 50 and the bottom member 20, including swivel support 24 and the dish 22, were made of clear polycarbonate. Polycarbonate can withstand steam sterilizing temperature of 260 degrees F or higher. The cover plate 50 and bottom member 20 were sterilized together by heat and steam, while the cover plate 50 was loosely fitted on the bottom member 20. When cooled, the cap and bottom were pressed close and the O-ring 36 sealed the interior 46 of the dish 22 in a sterile condition. Polycarbonate plastic has the benefit of being relatively heat resistant. Additionally, polycarbonate resists breakage, and is transparent allowing a user to see within device. Also, typical marker pens can be used label the devices directly on its surfaces.

Although the configuration of the dish 22 of the bottom member 20 and cover plate 50 of the embodiments illustrated in FIGS. 1 to 2B are substantially circular, it is intended in the present invention that other configurations be practiced as well. See FIGS. 3A and 3B illustrating alternative bottom member 20a & 20b and dish 22a & 22b configurations. Configurations for practice in the present invention in addition to those illustrated are known to and selectable by one of ordinary skill in the art.

While the above description contains many specifics, these should not be construed as limitations on the scope of the invention, but rather as exemplifications of one or another preferred embodiment thereof. Many other variations are possible, which would be obvious to one skilled in the art. Accordingly, the scope of the invention should be determined by the scope of the appended claims and their equivalents, and not just by the embodiments.

1. A stackable and pivotable petri dish system, comprising at least one petri dish having a bottom member, the bottom member further comprising:
a dish member having an opening for receiving and containing a material; and
a swivel support disposed adjacent to the dish member and perpendicular to the plane of the dish opening, the swivel adapted at its upper end to be received into the lower end barrel of a second like swivel support, with the bottom member being pivotable relative to an adjacent stacked bottom member via the action of the swivel support.

2. The stackable and pivotable petri dish system of claim 1, wherein the petri dish further comprising a cover plate for covering the dish opening of the dish on the bottom member.

3. The stackable and pivotable petri dish system of claim 1, further comprising a stacking rod to secure a plurality of petri dishes in a stacked configuration, the stacking rod having a first end passing serially through an axial bore in each of the stacked swivel supports from bottom to top and having a second end stopping within the axial bore of the bottom most petri dish, and a securing means disposed at the first end after it passes from the axial bore in the top most petri dish, the securing means preventing the individual swivel supports in a stack from separating from one another.
